(19) United States
(12) Patent Application Publication
(10) Pub. No.: US 2016/0302773 A1
(43) Pub. Date: Oct. 20, 2016

(54) DEVICE AND METHOD

(71) Applicant: ABON BIOPHARM (HANGZHOU) C., LTD., Hangzhou, Zhejianng (CN)

(72) Inventors: Gou Lijian, Hangzhou, Zhejiang (CN); Hu Haipeng, Hangzhou, Zhejiang (CN)

(21) Appl. No.: 15/101,355

(22) PCT Filed: Nov. 30, 2014
(86) PCT No.: PCT/CN2014/092625

§ 371 Date: Jun. 2, 2016

(30) Foreign Application Priority Data
Dec. 5, 2013 (CN) 201310651768.0
Dec. 5, 2013 (CN) 201310651785.4
Dec. 5, 2013 (CN) 201320796935.6

(51) Int. Cl. A61B 10/00; G01N 33/487

(52) U.S. Cl. CPC A61B 10/0051; G01N 33/487; A61B 2010/0009; A61B 2010/0003

(57) ABSTRACT

A device comprises a first cavity (300) and a second cavity (400), wherein the first cavity (300) is positioned in the second cavity (400), and may seal and compress the second cavity (400) so that the liquid in the second cavity (400) is discharged out quantitatively. The device may fully mix a buffer solution with a sample and achieves the function of integration of collection and test. Further, a collecting device also may achieve the quota-sampling function.

### FIELD OF THE INVENTION

The invention relates to a device and a method, particularly a device and method for collecting a sample, and a device and method for testing a sample after collecting the sample.

### BACKGROUND OF THE INVENTION

The following background art is to aid the reader in understanding the invention, but is not to be considered as the prior art.

Today, the abuse of illegal drugs has become a recognized and worsening social problem. In 2003, the United States Department of Health and Human Services (HHS) investigated and found that about 19.5 million Americans or 8.2 percent of the people over the age of 12 were taking illegal drugs. “The recently used illegal drug” refers to the illegal drug taken within one month before the investigation by HHS. Marijuana was found to be the most commonly taken illegal drug, accounting for 6.2% (14.6 million). Estimated 2.3 million people (1.0%) are now taking cocaine. 604,000 people are taking crack. 100 million people are taking hallucinogens. Estimated 119,000 people are taking heroin.

US Patents US2004/0184954 and US2004/0237674 have disclosed some other devices which collected saliva and detected whether the saliva contained ingredients of illegal drugs. The two patents both provide a device and method for collecting and testing saliva. In these devices, after the sample is collected into a collector, the sample in a suction component on a collector, on which external force is applied, is squeezed into a collecting cavity, and then tested. However, in the course that the sample collector and the collecting cavity are matched with each other and squeezed, an operator may touch the sample, causing contamination and infection. Furthermore, in the event that the sample shall be tested only after it is mixed with a buffer solution, such collecting devices become very un-convenient.

Additionally, in some tests, the sample needs to be treated and then can be tested. For example, the buffer solution is added into the sample, so that the sample is diluted and its viscosity is lowered, thus ensuring dialysis to be able to be smoothly carried out on a test strip. The problem, which is needed to be solved, is how to mix the buffer solution with the sample smoothly and fully to achieve convenient operation.

In other tests, in order to ensure the accuracy of tests, the amount of the sample needing to be tested shall be quantitative, so as to prevent too small amount of the sample from completing the test and prevent too much amount of the sample from producing Flooding phenomena, resulting in affecting the test accuracy of the results.

Therefore it is needed to have a better method and device for collecting and testing samples.

### SUMMARY OF THE INVENTION

The invention provides an improved device, the device may realize that a quantitative sample enters into a testing element, solving the problem that too much or too small amount of the sample causes incorrect or inaccurate test results; meanwhile, the device is still adapt to the test that the sample needs to be pretreated.

The invention provides a sample collecting device comprising a sample collector, wherein a sealed cavity is provided on the collector.

Preferably, the sealed cavity may be in liquid communication with a suction element on the collector through a passage.

Preferably, the device still includes a cover body, the cover body and the collector form the sealed cavity on the collector.

Preferably, the cover body is separably connected with the collector.

Preferably, the cover body and the collector, through a movable element on the collector, are matched with a through hole of the cover body, forming fixed connection and separation.

Preferably, when the movable element is positioned in a hole on the cover body, the cover body is fixedly connected with the collector; when the movable element is separated away from the hole on the cover body, the cover body and the collector are movable between them.

Preferably, the volume of the sealed cavity, through the movement between the cover body and the collector, is decreased or increased.

Preferably, with the movement between the cover body and the collector, the sealed cavity and the suction element of the collector are communicated with each other through the passage.

Preferably, the passage includes a hollow collecting rod, one end of the collecting rod is connected with the suction element, and the other end of the collecting rod is sealed and connected with the sealed cavity.

Preferably, a sealed film at the tip of the collecting rod is punctured with a convex in the sealed cavity, so that the sealed cavity and the hollow collecting rod are communicated with the suction element. In one particular example, the convex is positioned at the tip of the inner portion of the cover body while positioned in the sealed cavity.

Preferably, a buffer solution is provided in the sealed cavity.

The invention provides a device comprising a first cavity and a second cavity, wherein the first cavity is positioned in the second cavity; the first cavity may seal the second cavity; furthermore, the first cavity may compress the second cavity so that the liquid in the second cavity is discharged out quantitatively.

Preferably, the first cavity is in flexible connection with the second cavity. Preferably, when the first cavity is fixedly connected with the second cavity, the second cavity is not sealed; when the first cavity is separated away from the second cavity, the first cavity seals and compresses the second cavity.

Preferably, after the first cavity is separated away from the second cavity, the first cavity and the second cavity may be moved with each other.

Preferably, the first cavity has a first position and a second position on the second cavity. Preferably, when the first cavity is positioned at the first position, the first cavity seals the second cavity, forming the sealed cavity. Preferably, when the first cavity is positioned at the second position, the sealed cavity is compressed by the first cavity, the liquid in the sealed cavity is discharged out quantitatively.

Specifically, when the sample is not collected, the two cavities are fixedly connected with each other, so that the liquid sample is conveniently collected; after the sample is collected into the first cavity and the second cavity, the positions of the first cavity and the second cavity are changed, that is to say, the first cavity is rotated on the second cavity, so that the liquid is sealed and compressed in the second cavity and then discharged out of the second cavity, thus realizing the quota-sampling function. Specifically, after the two cavities are separated away from fixed connection, the first cavity is firstly rotated and moved downwards in the second cavity, when the first cavity reaches the first position, it seals the second cavity; more specifically, one small cavity is provided at the bottom of the second cavity, the cavity seals this small cavity and forms the sealed cavity containing the sample. The first cavity is rotated again and reaches the second position of the second cavity, and then the first cavity continues moving downwards, the volume of the small sealed cavity is compressed, wherein the sealed sample, according to the volume, discharges the corresponding amount of the liquid. As the distance between the first position and the second position is constant, the compressed volume of the sealed cavity is constant, the discharged amount of the liquid is constant, and quantitative sample is discharged out, thus achieving the quota-sampling function.

Preferably, a first convex of an upper rib of the first cavity is connected to and separated apart from the through hole of the second cavity, making the two cavities be connected and separated apart.

Preferably, the device still includes the sample collector, the sealed cavity is provided on the collector. Preferably, the sealed cavity may be in liquid communication with the suction element on the collector through the passage.

Preferably, the device still includes one cover body, the cover body and the collector form the sealed cavity on the collector. Preferably, the sealed cavity is positioned in the cover body, that is to say, the tip of the collector is positioned in the cover body and forms the sealed cavity on the collector. the cover body and the collecting rod are sealed and connected with each other, forming the sealed cavity in the cover body.

Preferably, the cover body is separably connected with the collector. That is to say, the two are in flexible connection.

Preferably, the cover body and the collector, through a movable element on the collector, are matched with the through hole of the cover body, forming fixed connection and separation.

Preferably, when the movable element is positioned in the hole on the cover body, the cover body is fixedly connected with the collector; when the movable element is separated away from the hole on the cover body, the cover body and the collector are movable between them.

Preferably, the volume of the sealed cavity, through the movement between the cover body and the collector, is decreased or increased.

Preferably, with the movement between the cover body and the collector, the sealed cavity and the suction element of the collector are communicated with each other through the passage.

Preferably, the passage includes a hollow collecting rod, one end of the collecting rod is connected with the suction element, and the other end of the collecting rod is sealed and connected with the sealed cavity.

Preferably, a sealed film at the tip of the collecting rod is punctured with the convex in the sealed cavity, so that the sealed cavity and the hollow collecting rod are communicated with the suction element. In one particular example, the convex is positioned at the tip of the inner portion of the cover body while positioned in the sealed cavity.

Preferably, the device still includes a test cavity, the test cavity is communicated with the sealed cavity of the second cavity.

Preferably, after the cover body and the collector which are fixedly connected are inserted into the first cavity, the collector is positioned in the first cavity and seals the first cavity; the cover body is positioned between the first cavity and the second cavity.

Preferably, the first cavity forces the movable element of the collector to be separated away from the through hole on the cover body.

Preferably, when the cover body moves with respect to the collector, the sealed cavity on the collector, which is formed by the cover body and the collector, is compressed, the convex at the tip of the cover body touches and punctures the sealed film at the tip of the collecting rod, the sealed cavity is communicated with a hollow cavity of the collecting rod.

Preferably, the buffer solution is provided in the sealed cavity, when the sealed cavity is communicated with the hollow cavity of the sample collecting rod, the buffer solution flows onto the suction element through the communicated hollow cavity. The buffer solution flows onto the suction element and is mixed with the sample on the suction element, completing the pretreatment of the sample.

Preferably, the cover body forces the first convex of the rib of the first cavity to be separated away from the through hole of the second cavity.

Preferably, the rib of the first cavity includes a second convex, the cover body forces the second convex to make the rib be retracted; after the rib is retracted, the first convex on the rib is separated away from the through hole in the second cavity. So, the first cavity is separated away from the second cavity, the two may be moved or rotated therebetween.

Preferably, when the sample collector together with the cover body is rotated in the first cavity and the second cavity, the suction element is compressed in the first cavity, the liquid flows into the first cavity and the second cavity; the first cavity together with the cover body and the collector is moved and reaches the first position of the second cavity, the first cavity seals the second cavity and forms the sealed cavity.

Preferably, when the first cavity together with the cover body is rotated and reaches the second position of the second cavity, the sealed cavity is compressed, the liquid in the sealed cavity enters into the test cavity quantitatively.

Preferably, the outer surface of the cover body and the inner surface of the second cavity have inter-occlusive threads.

On the other hand, the invention still provides a method for collecting a sample, the method provides a device for collecting a sample, the device includes a first cavity and a second cavity, the first cavity is positioned in the second cavity, both of the first cavity and the second cavity are separably connected and in liquid communication; the first cavity may seal and compress the second cavity so that the liquid in the second cavity may be discharged out quantitatively.

Preferably, the first cavity and the second cavity may be rotated with each other; so, the first cavity is rotated and reaches a first position of the second cavity, the first cavity seals the second cavity.

Preferably, the first cavity is rotated and reaches a second position of the second cavity, the sealed second cavity is compressed so that the liquid in the sealed second cavity is discharged out quantitatively.

Preferably, the device still includes a sample collector and a cover body which are movably connected; the sample collector includes a hollow collecting rod, the tip of the collecting rod is sealed with a sealed film, and the end thereof is connected with and communicated with a suction element; the cover body and the tip of the collecting rod form a sealed cavity on the collector.

Preferably, the sample collector and the cover body which are fixedly connected are inserted into the first cavity and the second cavity; the cover body is rotated so that the first cavity compresses a movable element on the sample collector to force the sample collector and the cover body to be separated away from the fixed connection.

Preferably, the cover body is rotated again so that the cover body and the sample collector are rotated with each other, the sealed cavity on the collector is compressed, the convex at the tip of the cover body touches and punctures the sealed film at the tip of the collecting rod, the sealed cavity is communicated with a hollow cavity of the collecting rod; the buffer solution in the sealed cavity flows onto the suction element through the communicated hollow cavity.

in the above course, the cover body and the collector are moved toward the lower portions of the first cavity and the second cavity while being moved therebetween, the collector is fixed relative to the position of the first cavity, that is to say, the collector is not moved.

Preferably, the cover body is rotated again so that the cover body compresses a rib on the first cavity to force the first cavity connected separably to be separated away from the second cavity.

Preferably, the cover body is rotated again so that the cover body together with the sample collector is rotated in the first cavity and the second cavity, the suction element is compressed in the first cavity, the liquid flows into the first cavity and the second cavity; the first cavity together with the cover body and the collector is moved and reaches the first position of the second cavity, the first cavity seals the second cavity and forms the sealed cavity.

Preferably, the cover body is rotated again so that the first cavity within the second cavity is moved and reaches the second position, the sealed cavity is compressed, the liquid in the sealed cavity quantitatively enters into a test cavity communicated with the sealed cavity.

### Effective Results

The invention provides a device and a method, which may fully mix a buffer solution with a sample and achieves the function of integration of collection and test, meanwhile, the collector still may achieve the quota-sampling function.

### BRIEF DESCRIPTION OF THE DRAWINGS

FIG. 1 is an exploded diagram of a device of the invention;

FIG. 2 is a diagram of one component (a cover body) of a device of the invention;

FIG. 3 is a diagram of the other component (a collector) of a device of the invention;

FIG. 4 is a diagram of components in FIG. 2 and FIG. 3 which are fixed together;

FIG. 5 is a diagram of the other component (a first cavity) of a device of the invention;

FIG. 6 is a diagram of the other component (a second cavity) of a device of the invention;

FIG. 7 is a diagram of a collecting cavity after components in FIG. 5 and FIG. 6 are assembled;

FIG. 8-FIG. 21 are diagrams of cross sections along directions A-A and B-B during the operation of a device of the invention, wherein directions A-A and B-B are vertical to each other;

FIG. 8 is a diagram of cross section A-A when a cover body of the invention covers and joins onto a first cavity and a second cavity;

FIG. 9 is a diagram of cross section B-B when a cover body of the invention covers and joins onto a first cavity and a second cavity;

FIG. 10 is a diagram of cross section A-A of one position where a cover body of the invention is rotated and reaches a first cavity and a second cavity (a first cavity compresses a movable element to make a collector be separated away from a cover body);

FIG. 11 is a diagram of cross section B-B of one position where a cover body of the invention is rotated and reaches a first cavity and a second cavity (a first cavity compresses a movable element to make a collector be separated away from a cover body);

FIG. 12 is a diagram of cross section A-A of the other position where a cover body of the invention is rotated and reaches a first cavity and a second cavity (a convex on a cover body punctures the top of a collecting rod);

FIG. 13 is a diagram of cross section B-B of the other position where a cover body of the invention is rotated and reaches a first cavity and a second cavity (a convex on a cover body punctures the top of a collecting rod);

FIG. 14 is a diagram of cross section A-A of another position where a cover body of the invention is rotated and reaches a first cavity and a second cavity (a cover body compresses a first cavity to force a first cavity to be separated away from a second cavity);

FIG. 15 is a diagram of cross section B-B of another position where a cover body of the invention is rotated and reaches a first cavity and a second cavity (a cover body compresses a first cavity to force a first cavity to be separated away from a second cavity);

FIG. 16 a diagram of cross section A-A of a first position where a first cavity of the invention is positioned in a second cavity (a suction element is compressed, and a first cavity seals a second cavity);

FIG. 17 a diagram of cross section B-B of a first position where a first cavity of the invention is positioned in a second cavity (a suction element is compressed, and a first cavity seals a second cavity);

FIG. 18 is a diagram of cross section A-A of a second position where a first cavity of the invention is positioned in a second cavity (a sealed cavity is compressed);

FIG. 19 is a diagram of cross section B-B of a second position where a first cavity of the invention is positioned in a second cavity (a sealed cavity is compressed);

FIG. 20 is a diagram of reverse cross section A-A of a device of the invention; and

FIG. 21 is a diagram of reverse cross section B-B of a device of the invention.

### DESCRIPTION OF REFERENCES

Collecting Device 800; Cover Body 100; Connection Hole 101 of Cover Body and Collector; Opening Hole 103 of Cover Body; Opening-Hole Plug 102; Lower Portion 110 of Cover Body; Convex 111 of Which Upper Portion and Lower Portion Are Joined; Liquid Cavity 107 Within Cover Body; Convex 106 at The Tip of Cover Body; Sealed Cavity 108 Formed By Cover Body and Collector; Opening 109 of Cover Body; Sample Collector 200; Sealed Film 201 at The Tip of Collector; Sealed Ring 202 Making Collector and Cover Body Be Matched; Hollow Cavity 210 Within Collector; Convex Groove 203 Received Within Cover Body; Collecting Rod (Hollow) 205; Movable Element 204 (Elastic Strip) Connected With Collecting Rod; Platform 2041 of Elastic Strip; Round-Shaped Disk 209 Receiving Cushion Ring 202; Suction Element 207; Cushion Ring 208 Making Collector and First Cavity Be Matched; Round-Shaped Disk 206 Receiving Cushion Ring 208; First Cavity 300; Second Cavity 400; Opening 305 of First Cavity; Upper Cavity 302of First Cavity; Middle Cavity of First Cavity 303; Lower Cavity 306 of First Cavity; Rib 310 On First Cavity; Second Convex 311 of Rib; First Convex 312 of Rib; Sealed Cushion Ring 310 Making First Cavity and Second Cavity Be Matched; Groove 304 Receiving Sealed Cushion Ring 301; Hole 307 Making First Cavity and Second Cavity Be Communicated; Inner Thread 402 of Second Cavity; Base 401; Convex Hole 405 Receiving Rib; Upper Cavity 403 of Second Cavity; Lower Cavity 404 of Second Cavity; Small Body 406 Within Second Cavity; Opening 4061 of Small Cavity; Hole 408 Making Second Cavity and Test Hole Be Communicated; Test Cavity 500, Upper Cover 502 of Test Panel; Lower Cover 501 of Test Panel; Test Element 600.

### DETAILED DESCRIPTION OF THE EMBODIMENTS

Sample Collector 200

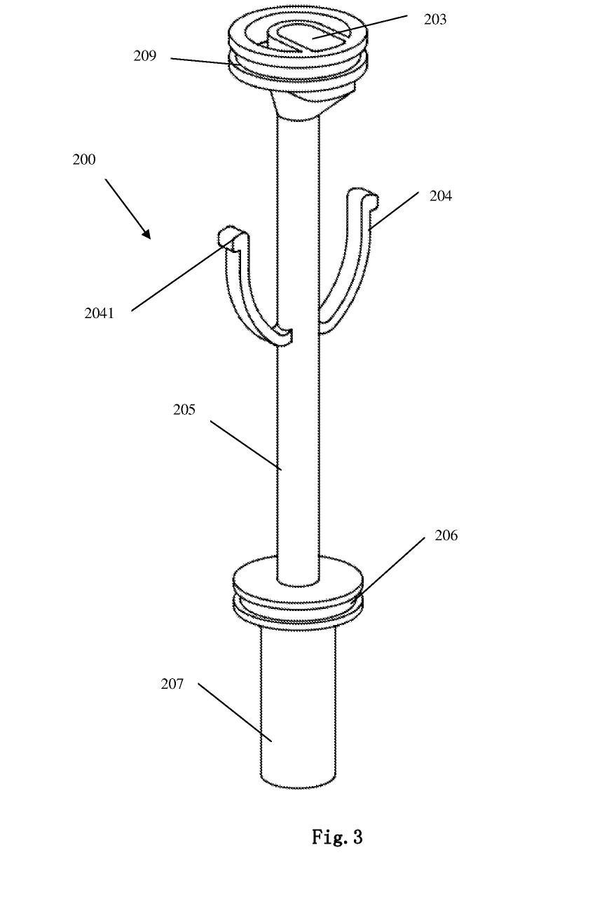

Generally, a sample collector 200 includes a suction element 207 and a collecting rod 205, the suction element 207 is positioned at one end of the collecting rod 205, as shown in FIG. 3. The suction element 207 is generally made of sponge or foam which is common and medical level in the prior art. However, the suction element may also be made of other materials, for example, cotton or paper, or any other material with water absorption capability. A holding portion is generally rigid, which facilitates the operation for the suction element. In some examples, the collecting rod is still taken as the holding portion, the holding portion may be made of common materials in the prior art, for example, plastic, wood, metal or paper panels. In one preferred example in the invention, in order that a sample on the suction element may be pretreated, the collector still includes a sealed cavity, which is used for receiving a buffer solution treating the sample. Preferably, under “specific conditions”, the sealed cavity is in liquid communication with the suction element. “The specific conditions” indicates the conditions that the sample is mixed with the buffer solution. Preferably, the sealed cavity is communicated with the suction element through “a passage”. In the specific way, the passage is the hollow collecting rod, the sealed cavity is positioned at one end of the hollow collecting rod, and the suction element is positioned at the other end of the hollow collecting rod. When the sample needs to be pretreated, the sealed cavity is communicated with the suction element through the collecting rod, the buffer solution flows onto the suction element.

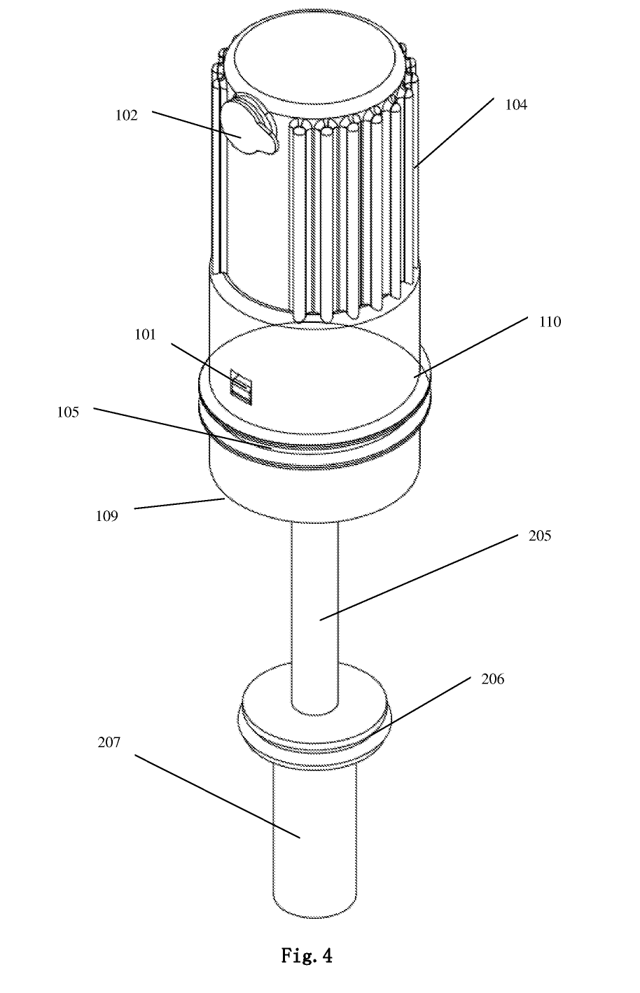

Preferably, the other end of the collecting rod 205 of the sample collector 200 is connected with the cover body 100, as shown in FIG. 4, the cover body may still have the function of the holding portion. In addition, the sealed cavity 108 is a hollow cavity where the collecting rod 205 and the cover body 100 are matched with each other and which forms in the cove body. The hollow collecting rod is communicated with the hollow cavity 108 of the cover body; furthermore, the hollow cavity of the collecting rod is communicated with the suction element 207. In the more specific example, the tip of the collecting rod has a sealed film 201, the hollow cavity of the collecting rod is separated away from the hollow cavity 108 of the cover body through the sealed film 201. In some specific examples, under “the specific conditions”, that is to say, when the sample needs to be treated, in the way that the sealed film 201 is punctured, the hollow cavity 205 of the collecting rod is communicated with the hollow cavity 108 of the cover body, as shown in FIG. 11. In some preferred examples, in order to guarantee that the collector and the cover body are in flexible connection with each other, a movable element is provided on the collector, with the matching between the movable element and the cover body, both of the collector and the cover body are fixed or separated away from each other. The movable element 204 may be moved within a certain range. Generally, the movable element has elasticity, its one end is connected on the collecting rod, and its other end is moved when it is applied by pressure. In one specific example, the movable element is an elastic strip 204 used for connecting a collecting cavity 300. In one specific example, the two elastic strips 204 are symmetrically positioned at the middle of the collecting rod 205. The elastic strip 204 is in hook shape, and its position is changed when being applied by pressure, that is to say, when the sample collector 200 is positioned in the collecting cavity 200, and when the elastic strip 204 is applied by the pressure from the collecting cavity 300, the elastic strip 204 springs back automatically, so that the collector 200 may be moved freely in the collecting cavity 300. In the other specific example, the collector 200 is fixedly connected with the cover body 100 through the elastic strip 204. When the elastic strip 204 has unknown offset after being stressed, the elastic strip 204 leaves the cover body 100, the cover body 100 and the collector 200 are separated away from the fixed connection.

Cover Body 100

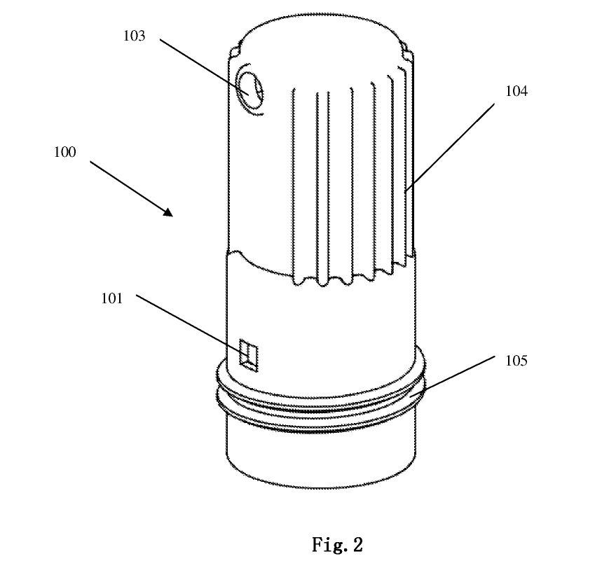

The invention still includes a cover body 100 used for sealing a collecting cavity body 300, as shown in FIG. 2. In one specific example, the cover body 100 is connected with and communicated with a collector 200. In a more specific example, the cover body 100 is in flexible connection with the collector 200. During one operation, the cover body 100 is fixedly connected with the collector 200; while during the other operation, the cover body 100 is not fixedly connected with the collector 200. Preferably, a through hole on the cover body is matched with a movable element on the collector and makes both of them be fixed or separated apart, a specific example, the number of the through holes is two and identical to the number of the movable elements on the collector. When the collector is inserted into the cover body, a cylinder at the tip of the collector is matched with a side wall in the cover body and they form sealing, that is to say, a sealed cavity 204 is formed, the collector continues to be moved into the cover body deeply, one opening end of the movable element on a collecting rod touches the through hole on the cover body, enters into the through hole and is fixed. At this time, the collector is fixed with the cover body, the volume of a sealed cavity on the collecting rod is also fixed.

Preferably, in order to guarantee that the sealed cavity is communicated with a suction element if necessary, a convex is designed in the sealed cavity on the cover body (that is to say, in the inner portion of the cover body), one end of the convex is sharp and may puncture materials such as aluminum foil, paper, plastic film, and the like. When a sample needs to be pretreated, the convex touches and punctures a sealed film at the tip of the collecting rod, thus making the sealed cavity and a hollow cavity of the collecting rod be communicated with each other. In a specific example, with the mutual rotation between the cover body and the collector, the sealed cavity is compressed and its volume becomes small, the distance between the tip of the cover body and the tip of the collecting rod is decreased, the convex at the tip of the cover body touches, compresses and punctures the sealed film at the tip of the collecting rod, so as to realize the communication between the cavities.

In one example, a thread 105 on an outer side wall of the cover body 100 is matched with the collecting cavity. In some other examples, one liquid storage cavity 107 is provided at the tip of the inner portion of the cover body, the liquid storage cavity 107 is used for storing excessive tested liquid sample. In a more specific way, the cover body 100 is still provided with one opening 106 at the corresponding place of the liquid storage cavity 107, the liquid in the liquid storage cavity 107, through the opening 103, may be taken out to be tested and confirmed again.

Collecting Cavity (A First Cavity, A Second Cavity)

Generally, a collecting cavity is used for receiving liquid sample, is communicated with a test cavity 500 and finally transports the sample onto a test element 600 to be tested. In a specific example, the collecting cavity is enclosed by the side wall and the bottom, and has one opening for receiving the sample. In the invention, in order to realize a quota-sampling function, the collecting cavity is a split-type structure and consists of two cavities (namely, a first cavity 300, a second cavity 400).

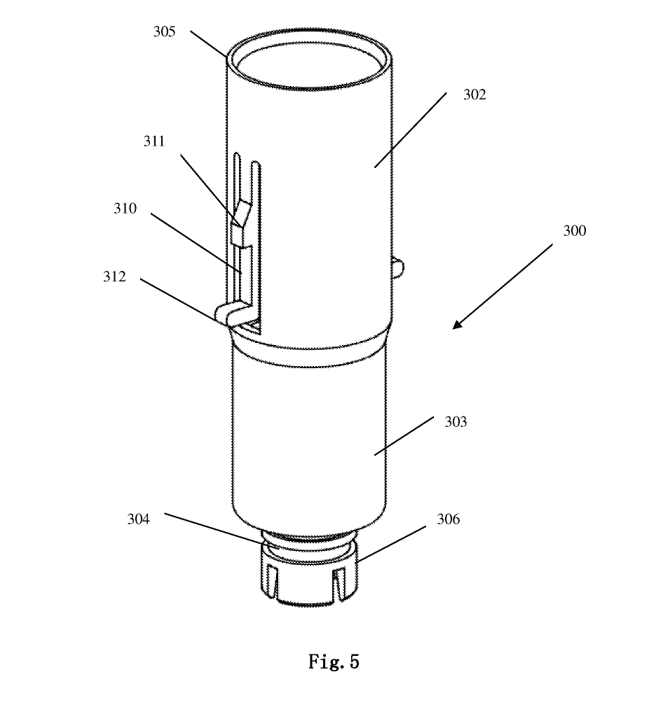

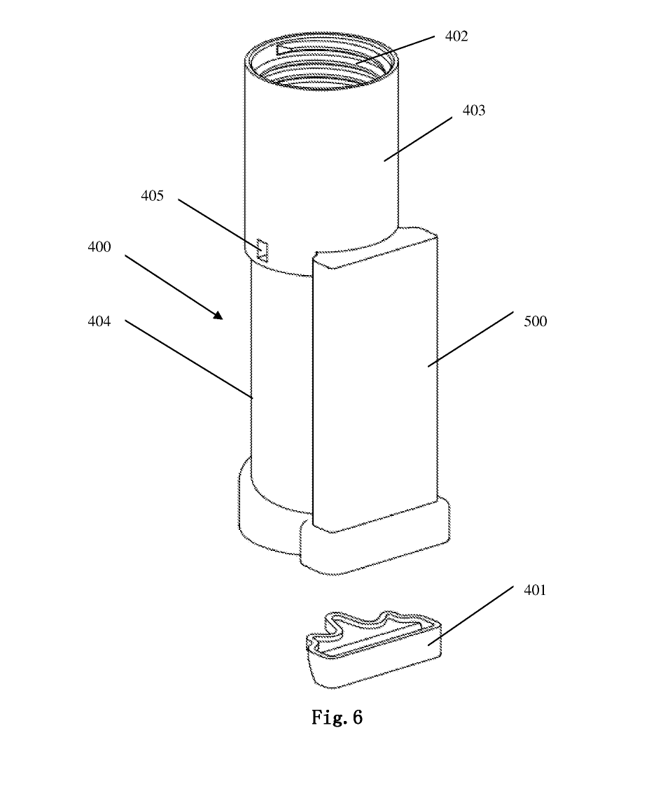

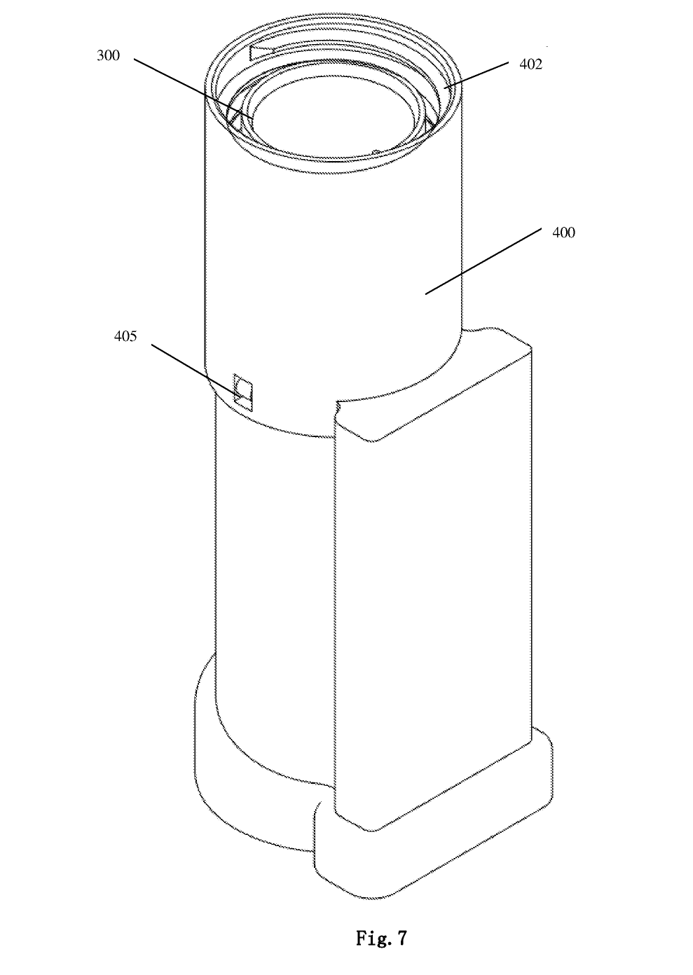

As shown in FIGS. 5 and 6, the first cavity 300 is positioned in the inner portion of the second cavity 400, furthermore, two the cavities are communicated with each other, as shown in FIG. 7. Specifically, a through hole is provided at the bottom of the first cavity, through the through hole 307, the first cavity is communicated with the second cavity. Preferably, the through hole 307 is sealed in the first cavity and sealed when it compresses the second cavity. As shown in FIG. 5, one small cavity 306 is provided at the bottom of the first cavity, the opening of the small cavity 306 is opposite to the direction of the first cavity, that it, it is downward, separated away from a middle cavity on the first cavity and only communicated with the middle body through the through hole; in the second cavity, the space of the bottom of the cavity is obviously decreased and forms another small cavity 406; when the first cavity seals the second cavity, the small cavity 306 is positioned in the small cavity 406 at the bottom of the second cavity, furthermore, the top of the small cavity 306 of the first cavity seals the lower cavity 406 of the second cavity and forms the sealed cavity. When the first cavity compresses the second cavity, the small cavity 306 of the first cavity is moved in an inner cavity 406 of the second cavity toward the bottom thereof, the top of the small cavity 306 compresses and seals the cavity 406. More preferably, when the sealed cavity 406 has liquid therein, the liquid is compressed.

In one example, the first cavity 300 is separably connected with the second cavity 400, that is, they are in flexible connection, under one state, both of two the cavities are fixed together, under the other state, both of two the cavities are separated away from each other, and movable with each other. In another specific example, the first cavity 300 and the second cavity 400, through a first convex of a rib of the first cavity, are connected and fixed. Preferably, the rib is elastic to facilitate the rib and the convex thereon to be retracted. In some specific examples, the rib is positioned on the first cavity and becomes one portion of the first cavity, but only one end of the rib is connected with the first cavity and the rest is hollow out. Such a structure may effectively enhance the elasticity, when the rib is applied by pressure, its position is changed.

In some other examples, when the first cavity is positioned at one position (a first position) in the second cavity, the first cavity and the inner side wall in the second cavity form sealing and seal the second cavity, more specifically, one sealed cavity 406 forms between the first cavity and the second cavity, the sealed cavity is connected with the first cavity through the through hole 307, after the through hole 307 is sealed, the sealed cavity 406 and the test cavity 500 form a sealed space, as the sealed space has quantitative air, when the air in the sealed space is compressed, the liquid in the sealed cavity enters into the test cavity quantitatively, thus realize the quota-sampling function.

A structure or technical terms used in the invention is further described in the followings. In the following detailed description, the reference text of the figures is one portion of the followings and described with examples by the way that the invention may be carried out by particular and practicable embodiments. We don't exclude that the invention may also carry out other embodiments and its structure may be changed without departing from the use protection scope of the invention.

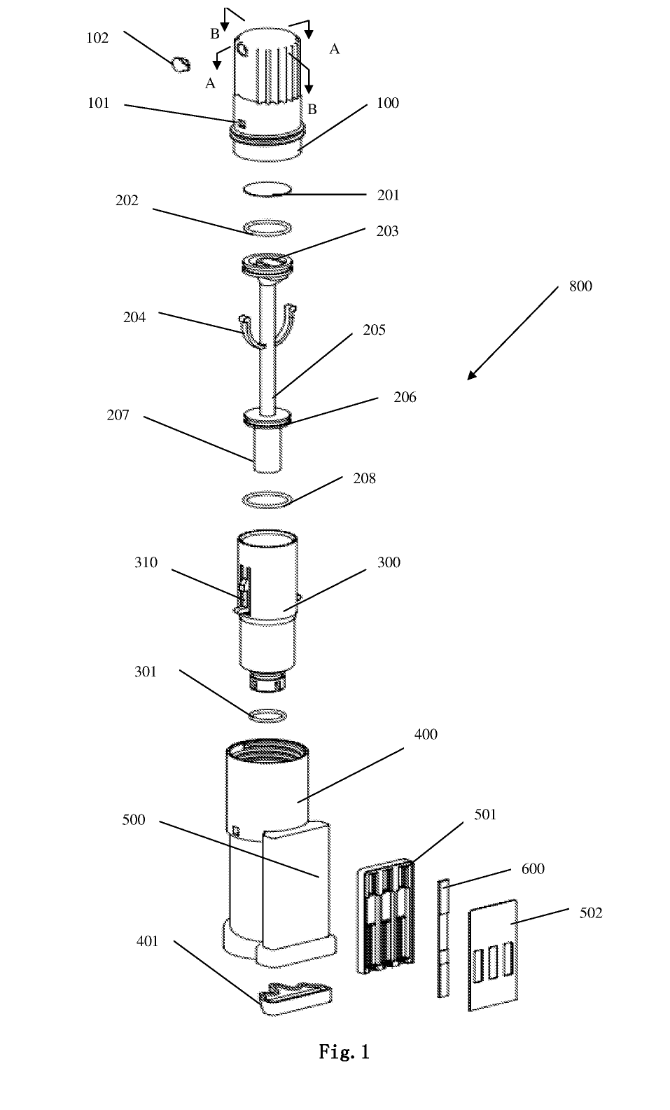

As shown in FIG. 1, the invention specifically relates to a collecting device 800, the collecting device includes four components: a cover body 100, a collector 200, a first cavity 300 and a second cavity 400. With the matching of four the components, the functions, such as the mixture of a sample and the buffer solution of the device, the collection of the mixed solution of the sample, the quota-sampling of the sample, the test of the sample and so on, are realized.

As shown in FIG. 2, the cover body 100 has two portions, that is, an upper portion 104 of the cover body; a lower portion 110 of the cover body; wherein the upper portion 104 is smaller than the lower portion 110, therefore, a convex round-shaped platform 111 is provided at the joining place between the upper portion 104 and the lower portion 110. The inner side of the upper portion of the cover body is provided with one liquid storage cavity 107, the cavity 107 is communicated with the outside through one opening 103, the liquid storage cavity 107 is used for storing the tested residual sample, so as to facilitate re-test. The opening 103 includes one opening-hole plug 102, the liquid storage cavity 107 and the cover body 100 are sealed through the opening-hole plug. In addition, two symmetrical holes 101 are still provided at the middle place of the lower portion of the cover body 110, the hole 101 is used for connecting the collector 200 and making the two be fixed. In some specific examples, one convex 106 is still provided at the tip of the inner portion of the cover body 100, as shown in FIG. 9. Similarly, in order to realize the matching of the cover body 100 and the collecting cavity, and in order that the cover body may seal the collecting cavity, a thread 105 is still provided at the outside of the lower portion of the cover body 100.

As shown in FIG. 3, a sample collector 200 consists of a collecting rod 205 and a suction element 207 connected with one end of the collecting rod, furthermore, the other end of the collecting rod 205 is provided with one round-shaped surface 209 for forming the sealing with the cover body 100. A groove and a sealed ring 202 within the groove are provided at the side wall of the round-shaped surface 209 contacting the cover body. The diameter of the round-shaped surface 209 of the collecting rod is identical to the inner radius of the upper portion 104 of the cover body, therefore, when the collector 200 is connected with the cover body 100, the round-shaped surface 209 of the collecting rod is closely matched with the inner portion of the upper portion of the cover body, and makes a cushion ring 202 on a round-shaped surface and the inner side wall of the upper portion of the cover body form the sealing, hence, one sealed cavity 108 forms at the tip of the collecting rod 205 and the inner portion of the cover body. In one specific example, the collecting rod 205 of the collector is a hollow structure, through the hollow portion, the tip of the collector is in liquid connection with the liquid at the bottom, that is, it is in liquid connection with the suction element 207 at the bottom end. At the tip of the collector 205, one sealed film 201 seals the hollow portion of the collector. The sealed film may be made of plastic, and film or paper with a sealing function, etc. and shall have two functions, that is, it can be sealed with liquid and be punctured with a shape. In one example, the tip of the hollow portion of the collecting rod still is provided with a groove 203 receiving a convex in the cover body, when the cover body is connected with the collector, the convex 106 on the cover body punctures the sealed film 201 at the tip, and is positioned in the groove 203. In some specific examples, two symmetrical elastic strips 204 are still provided on the collector 200, the elastic strip 204 has a retraction function when it is applied by pressure. The elastic strip 204 is in overhead-hook shape, and has one small platform at the end thereof. On the collector 200, in order that the suction element 207 may be sealed in the collecting cavity, a disk 206, of which the inner diameter is the same as that of a collecting cavity, is still provided on the suction element 207, the groove is designed at the side of the disk, a sealed ring 208, which may seal the collecting cavity, is provided in the groove.

As shown in FIG. 4, when the cover body joins the collector, a round-shaped surface at the tip of the collector, from the opening 109 of the cover body, enters into the cover body, the round-shaped surface moves from the lower portion of the cover body to a round-shaped platform at the lower portion thereof, starts to sealing the upper portion 104 of the sealed cover body, and continues to push the collector 200 until the platform 2041 of the elastic strip on the collector is positioned on a through hole 101 of the middle of the cover body, at this time, both of them are fixed, when the collector 200 is positioned in the cover body 100, one sealed cavity 108 is formed between the two, the cavity 108 is used for receiving the buffer solution.

As shown in FIG. 5, the first cavity 300 includes an upper cavity 302, a middle cavity 303 and a lower cavity, moreover, the diameter of three the cavities is decreased from top to bottom successively. The middle cavity 303 and the lower cavity 306 are separated away from each other through a baffle, the through hole 307 is provided on the baffle to guarantee the communication among three the cavities. Two symmetrical ribs 310 are provided on the upper cavity 302. One end of the rib 310 is connected with the cavity 300, and the rest is not connected with the cavity 300, when the rib 310 is applied by pressure, the position of the rib 310 is offset. Two the convexes (a second convex 311 and a first convex 312) are provided on the rib 310. The first convex 312 is used for connecting the cavity with the second cavity 400. In one example, the connection place of the middle cavity 303 and the lower cavity 306 is provided with the groove 304 and the sealed cushion ring 301 in the groove, when the first cavity is positioned in the inner portion of the second cavity, the cushion ring 301 seals the inner side wall of the second cavity.

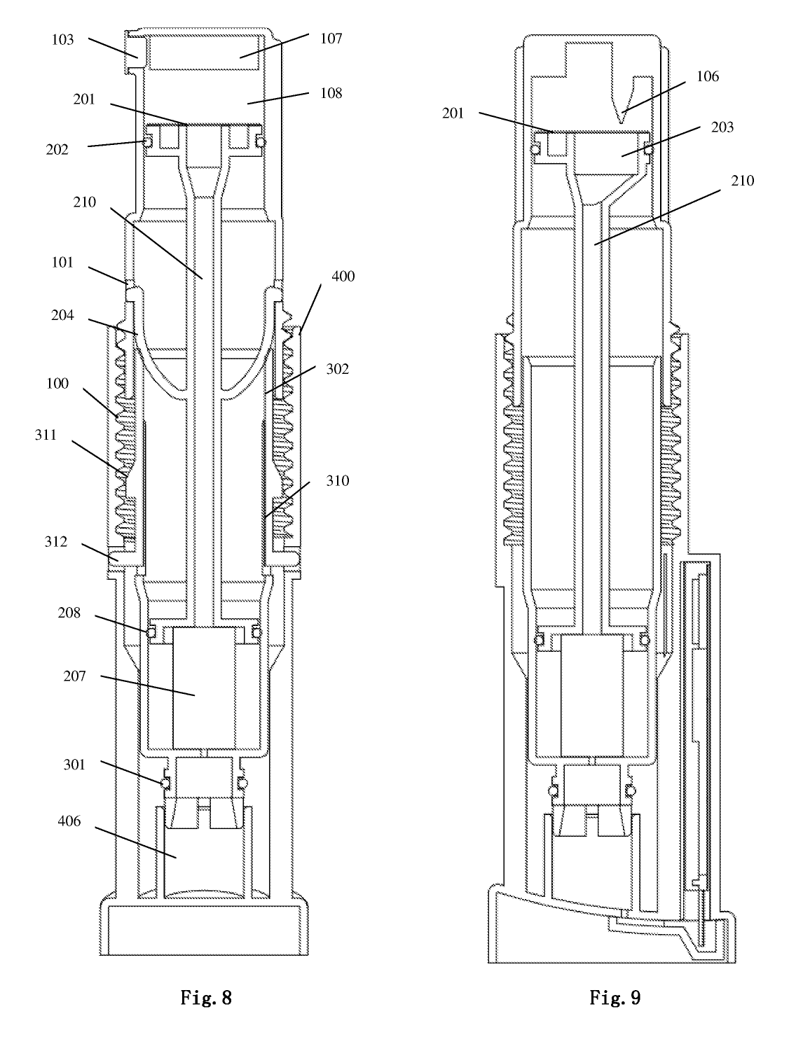

In FIG. 6, the second cavity 400 comprises the upper cavity 403 and the lower cavity 404, the tip of the inner side of the upper cavity is provided with the thread 402, the thread 402 is corresponding to the thread 105 at the outer side of the cover body. A sample test cavity 500 is still provided at the outside of the second cavity, the test cavity 500 is communicated with the second cavity 400 through the through hole 408, so as to guarantee the liquid in the second cavity 400 to pass through the through hole 408 and enter into the test cavity 500. A base 401 is provided at the bottom of the second cavity. A hole 405, which is connected with the rib 310 of the first cavity, is provided on the upper cavity 403, when the first cavity 300 is positioned in the second cavity 400, the rib on the first cavity is positioned on the hole 405 in the second cavity, at this time, the first cavity 300 is fixed in the second cavity 400, as shown in FIG. 7. As shown in FIG. 8, one small cavity 406 is still provided at the bottom of the inner portion of the lower cavity 404 of the second cavity, the inner diameter of the cavity is identical to the outer diameter of the lower cavity 306 of the first cavity, so as to receive the lower cavity 306 and form the side-wall sealing together with the sealed ring 301 on the lower cavity 306.

As shown in FIG. 6, a test element 600 may be positioned in the test cavity 500 for testing the sample flowing into the test cavity. In one specific example, one test panel is placed in the test cavity, the test panel, through the covering and closing of an upper panel 501 and a lower panel 502, places the test element 600 therein.

The specific operation of the collecting device 800 is described in details in the followings. FIG. 8-FIG. 21 show the operation step of a sample collecting device and the specific state of the collecting device after each operation by a way of a cross-section diagram. Under the initial state, the cover body 100 and the collector 200 are connected with each other and fixed through an elastic strip 204. At this time, the sealed cavity 108 has the buffer solution therein; the first cavity 300 is positioned in the second cavity 400 and they are fixedly connected with each other through the rib 310. At this time, the upper cavity 302 of the first cavity and partial of the middle cavity 303 are positioned in the upper cavity 403 of the second cavity, the rest is positioned in the lower cavity 404 of the second cavity; when the suction element 207 on the sample collector collects the sample (for example, saliva sample), the cover body 100 is covered and closed on the collecting cavity, at this time, the sample collector 200 is positioned in the first cavity 300, the opening 109 of the cover body is positioned between the first cover body 300 and the second cavity 400, as shown in FIG. 8, meanwhile, the first cavity 300 is still positioned between the cover body 100 and the sample collector 200; at this time, the buffer solution used for buffer is placed in the sealed cavity 108 connected and formed by the cover body 100 and the collector 200;furthermore, as shown in FIG. 9, the convex 106 at the tip of the cover body is positioned in the sealed cavity 108, there is a certain distance between the convex 106 and the sealed film 201 at the tip of the collector.

Then, the cover body 100 is rotated, the thread 105 outside of the cover body and the thread 402 in the inner portion of the second cavity are occluded with each other, so that the cover body 100 is moved downward along the first cavity 300 and the second cavity 400 and reaches the second position, as shown in FIG. 10, at this time, the sample collector 200 and the cover body 100 are fixed together through the elastic strip 204, the sample collector is moved with the movement of the cover body 100, therefore, the elastic strip 204 on the sample collector starts to touch the opening 305 of the first cavity, as the strip 104 has elasticity and springs back when it is pressed by the opening 305 of the first cavity, so that the platform 2041 on the elastic strip starts to be away from the opening hole 101, therefore, the sample collector 200 and the cover body 100 are separated away from the fixed connection, as shown in FIG. 10. As shown in FIG. 11, as the collector 200 and the cover body 100 are fixedly connected with each other, the cover body 100 is not moved with respect to the collector 200, therefore, it is not changed either that there is a distance between the convex 106 of the cover body and the sealed film 201.

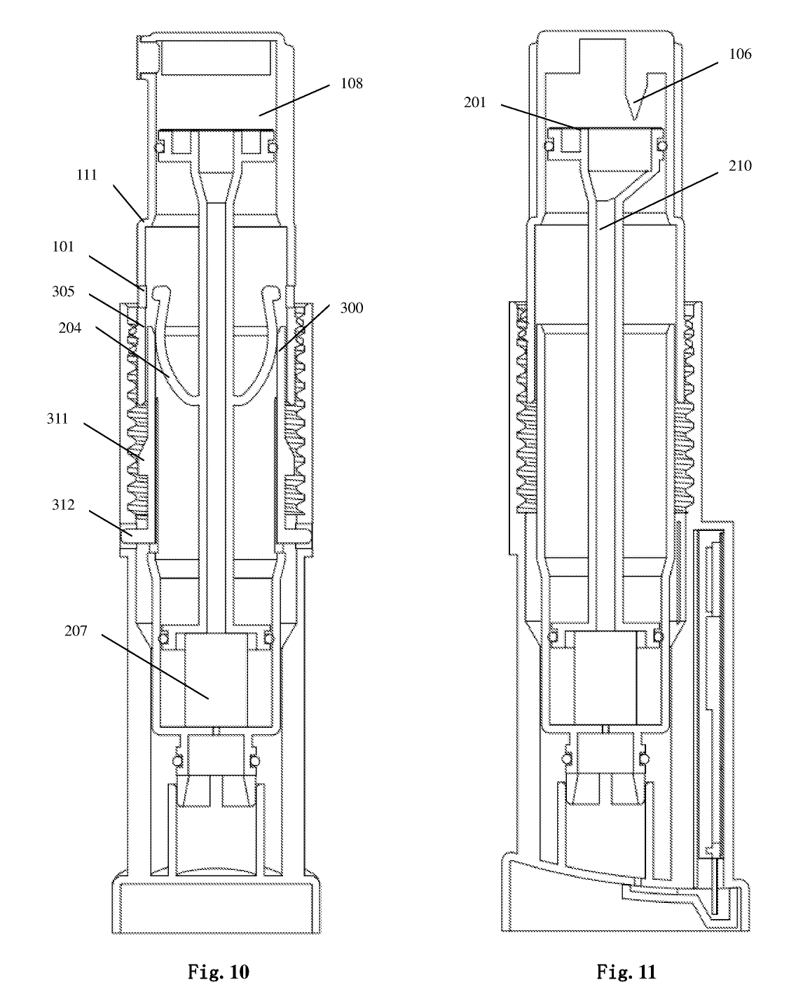

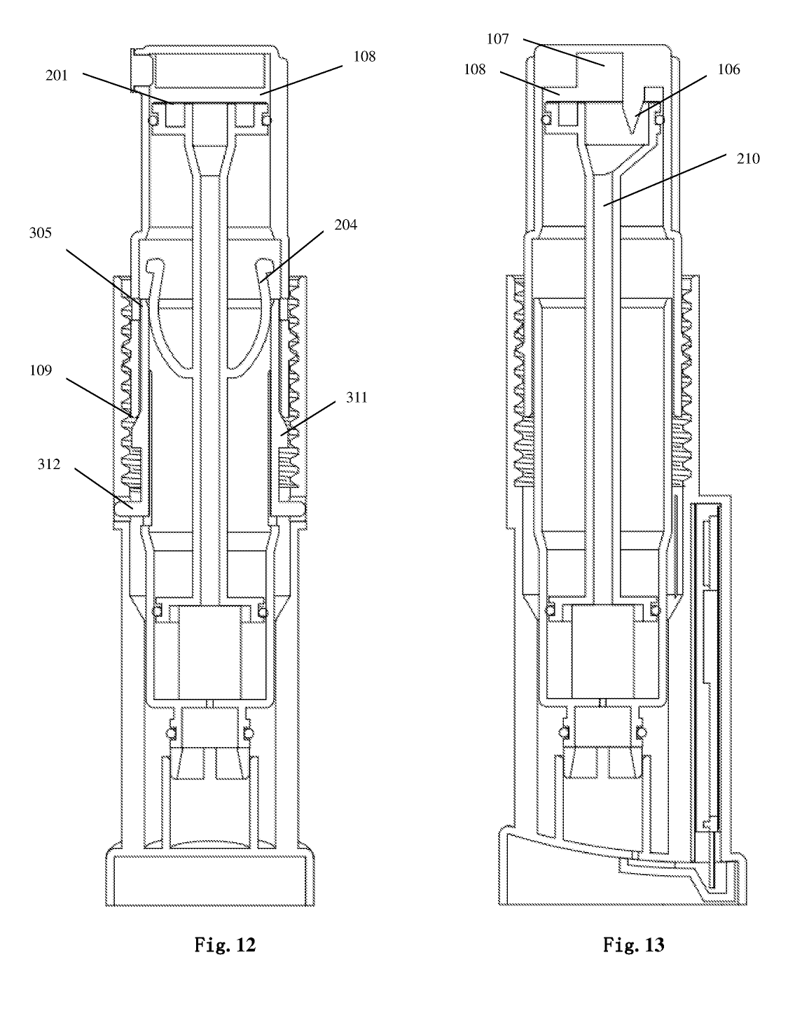

The cover body 100 is rotated again, the cover body 100, in a thread occlusion way, continues to move downwards along the first cavity 300 and the second cavity 400, so that the cover body 100 reaches the second convex 311 of the rib in the first cavity. As shown in FIG. 12, in the process of this movement, as the sample collector 200 and the cover body 100 are separated away from the fixed connection, during the continuous downward movement of the cover body 100, the sample collector 200 is not moved with the cover body 100 any more, that is, the position of the sample collector 200 is not changed relative to the first cavity 300 and the second cavity 400, that is, the opening of a first collecting cavity touches the center of the elastic strip of the collector. The sample collector 200 is not moved with respect to the cover body 100; with the downward movement of the cover body 100, the sealed cavity 108 between the cover body 100 and the sample collector 200 is decreased, as shown in FIG. 12, therefore, the convex 106 of the cover body in the sealed cavity approaches gradually the sealed film 203 at the tip of the sample collector, and finally reaches and punctures the sealed film 203, as shown in FIG. 13. The sealed cavity 108 is communicated with the hollow cavity 210 of the sample collector, the buffer solution in the sealed cavity 108 flows into the hollow cavity 210 of the sample collector, finally reaches the suction element 207 on the sample collector and is fully mixed with the sample collected on the suction element 207, the sample on the suction element is pretreated. The big-funnel-shaped groove 203 for receiving the convex 106 of the cover body is at the tip of the hollow cavity 210 of the sample collector, so that the liquid in the sealed cavity 108 conveniently flows into the hollow cavity 210 of the sample collector.

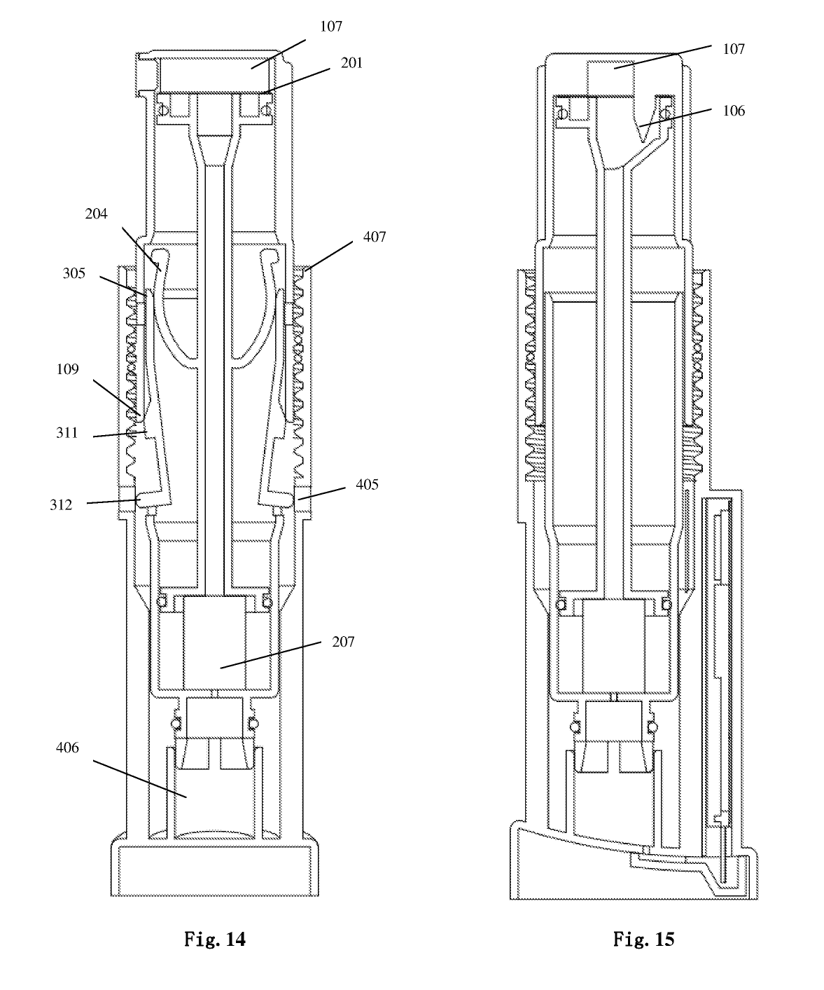

The cover body is rotated again, the cover body 100 continues to be moved downwards along the first cover body 300 and the second cavity 400. As shown in FIG. 14, as this time, during the cover body is moved downwards between the first cavity 300 and the second cavity 400, as the second convex 311 on the rib touches the opening 109 of the cover body, during the cover body 100 continues to be moved downwards, the opening of the cover body 109 is pressed on the second convex 311, so that the second convex 311 springs back to make the whole rib 310 be pressed inwards. The first convex 312 on the rib is also forced to be retracted in the first cavity 300, so that the first convex 312 is separated away from the through hole 405 of the second cavity, as shown in FIG. 14. Once the first convex 312 is separated away from the through hole 405, the first cavity 300 and the second cavity 400 are not fixedly connected with each other any more, both of them may have position change. In addition, as shown in FIG. 15, the cover body is moved to the tip of the sample collector, that is, the sealed cavity 108 is not compressed, that is, when the cover body is moved downwards again, the collector is forced to be moved together.

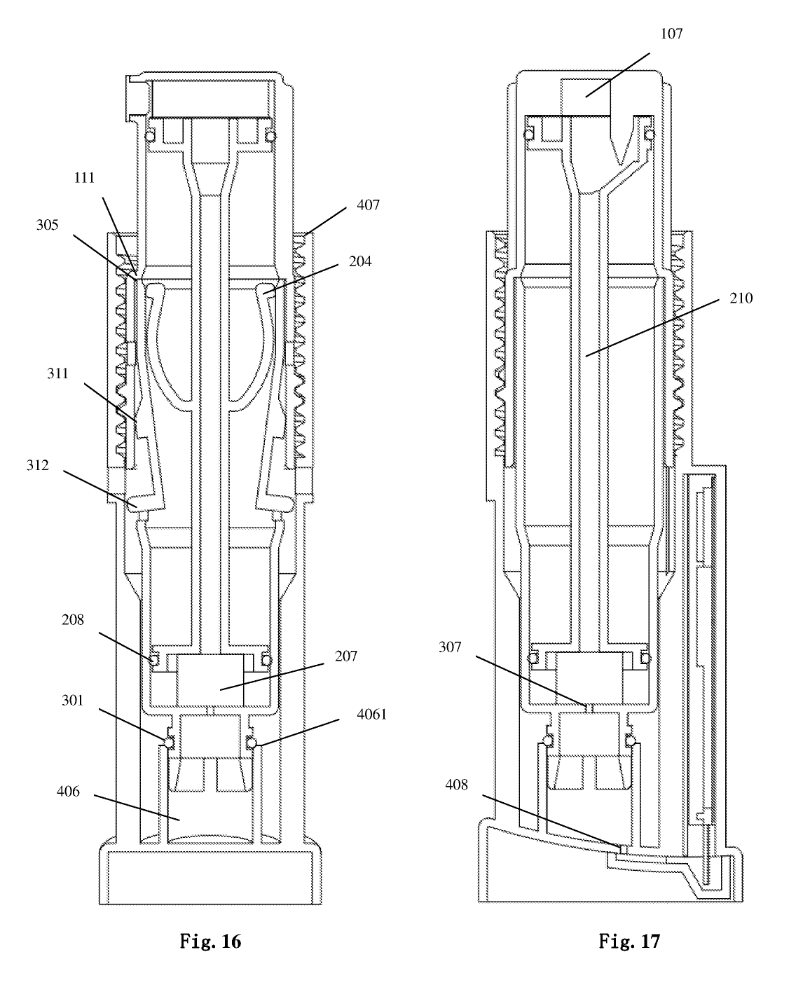

The cover body 100 is rotated again, the cover body continues to be moved downwards along the first cover body 300 and the second cavity 400, at this time, as the collector 200 has reached the tip of the cover body 100, both of them are moved together. During the movement, the opening 305 of the first cavity reaches the tip of the elastic strip 204 of the collector, reaches the convex 111 where the upper portion and the lower portion of the cover body are intersected, is blocked by the cover body 100 and is not moved any more, as shown in FIG. 16. Moreover, as the distance between the suction element 207 on the collector and the collector is shortened due to the first cavity, the space of the suction element 207 in the middle cavity 303 of the first cavity is decreased and the element 207 is hence compressed, the mixed liquid on the element 207 flows into the middle cavity 303 in the first cavity, as shown in FIG. 16 and FIG. 17. Meanwhile, as the first convex 312 is separated away from the hole 405 of the second cavity, the first cavity 300 and the second cavity 400 are separated way from the fixed connection, after the cover body is rotated, the first cavity 300 firstly reaches the convex 111 of the cover body, is blocked and is fixed, when the cover body 100 continues to be rotated, the first cavity 300 is driven to be moved downwards in the second cavity 400, so that the first cavity 300 is moved downwards in the second cavity 200, and finally the first cavity reaches the first position of the second cavity, at this time, the sealed cushion ring 301 on the lower cavity of the first cavity reaches the opening 4061 of the inner cavity 406 in the second collecting cavity and seals the inner cavity 406, as shown in FIG. 16, at this time, the lower cavity 306 of the first cavity 300 and the inner cavity 406 of the second cavity form one sealed cavity, the sealed cavity is communicated with the middle cavity 303 only through the through hole 307, and communicated with the test cavity 500 through the through hole 408, as shown in FIG. 17.

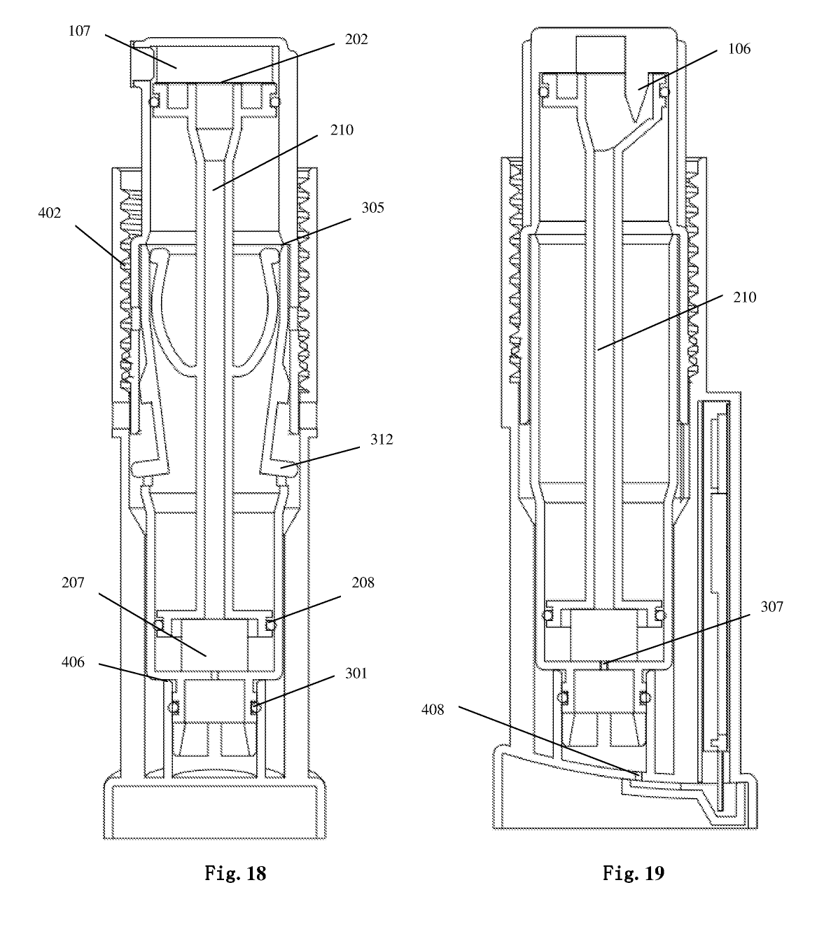

The cover body is rotated again, at this time, the sample collector 200 and the first cavity 300, together with the cover body 100, continue to be moved in the second cavity 400, so that the first cavity 300 reaches the second position of the second cavity. As shown in FIG. 18 and FIG. 19, during the movement of the cover body, the suction element is compressed again, the mixed liquid thereon is discharged out and flows into the middle cavity 303, and flows into the sealed cavity through the through hole 307, with the compression of the suction element, the liquid in the suction element is exhausted and blocks the through hole 307, furthermore, the cushion ring 208 on the suction element seals the side wall of the middle cavity 303, so that the sealed cavity and the test cavity 500 form a sealed space, the sealed air and the sealed liquid in the sealed space both have fixed amount. During the cover body 100 continues to be moved, the first cavity 300 continues to be moved into the second cavity 400 deeply, so that the lower cavity 306 of the first cavity is completely moved into the inner cavity 406, furthermore, the sealed space 406 is further compressed, so that the air pressure in the space is increased, therefore, the certain amount of the liquid in the inner cavity 406 flows into the test cavity 500 through the through hole 408 and reaches the test element, thus completing the test of the sample. The amount of the liquid in the test cavity is determined by rotating the cover body to compress the sealed space.

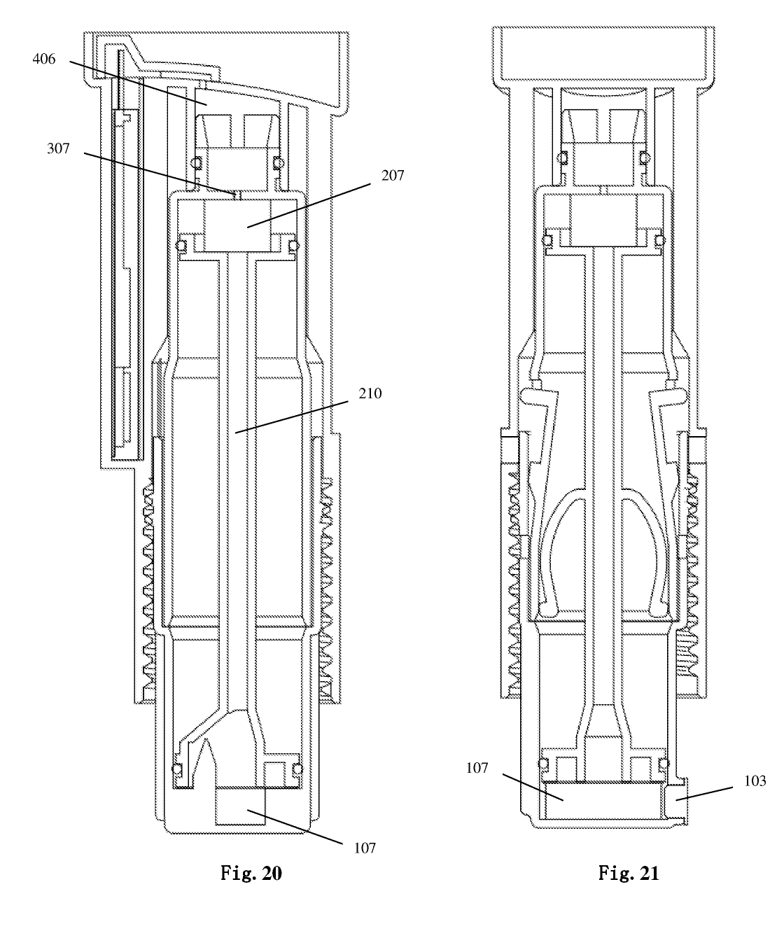

After the test, as shown in FIG. 20 and FIG. 21, the collecting device 800 is reversed, so that the excessive sample in the test cavity 500 may flow back to the collecting cavity, at this time, the cover body 100 is circled round, so that the pressure of the sealed space is increased, and finally negative pressure is formed, thus facilitating the backflow of the liquid, the backflow liquid flows onto the suction element 207 through the through hole 307, flows into the hollow cavity 201 through the suction element and finally reaches the liquid storage cavity 106. When the sampling is needed, the opening-hole plug 102 is opened, and the sample is taken out from the through hole 103.

1. A device comprising a first cavity and a second cavity, wherein the first cavity is positioned in the second cavity; the first cavity may seal the second cavity; furthermore, the first cavity may compress the second cavity so that the liquid in the second cavity is discharged out quantitatively.

2. A device according to claim 1, wherein the first cavity is in flexible connection with the second cavity.

3. A device according to claim 2, wherein after the first cavity is separated away from the second cavity, the first cavity and the second cavity may be moved with each other.

4. A device according to claim 1, wherein: the first cavity has a first position and a second position on the second cavity.

5. A device according to claim 4, wherein when the first cavity is positioned at the first position, the first cavity seals the second cavity, forming the sealed cavity.

6. A device according to claim 4, wherein when the first cavity is positioned at the second position, the sealed cavity is compressed by the first cavity, so that the liquid in the cavity is discharged out quantitatively.

7. A device according to claim 2, wherein a first convex of an upper rib of the first cavity is connected to and separated away from a through hole of the second cavity, making the two cavities be fixedly connected and separated apart with each other.

8. A device according claim 1, wherein the device still comprises a sample collector provided thereon with the sealed cavity; the sealed cavity may be in liquid communication with a suction element on the collector through a passage.

9. A device according to claim 8, wherein the device still comprises a cover body, the cover body and the collector form the sealed cavity on the collector.

10. A device according to claim 9, wherein the cover body is separably connected with the collector.

11. A device according to claim 10, wherein the cover body and the collector, through a movable element on the collector, are matched with the through hole of the cover body, forming fixed connection and separation.

12. A device according to claim 11, wherein when the movable element is positioned in a hole on the cover body, the cover body is fixedly connected with the collector; when the movable element is separated away from the hole on the cover body, the cover body and the collector are movable between them.

13. A device according to claim 11, wherein the volume of the sealed cavity, through the movement between the cover body and the collector, is decreased or increased.

14. A device according to claim 11, wherein with the movement between the cover body and the collector, the sealed cavity and the suction element of the collector are communicated with each other through the passage.

15. A device according to claim 8, wherein the passage comprises a hollow collecting rod, one end of the collecting rod is connected with the suction element, and the other end of the collecting rod is sealed and connected with the sealed cavity.

16. A device according to claim 15, wherein a sealed film at the tip of the collecting rod is punctured with a convex in the sealed cavity, so that the sealed cavity and the hollow collecting rod are communicated with the suction element.

17. A device according to claim 1, wherein the device still comprises a test cavity, the test cavity is communicated with the sealed cavity of the second cavity.

18. A device according to claim 1, wherein after the cover body and the collector which are fixedly connected are inserted into the first cavity, the collector is positioned in the first cavity and seals the first cavity; the cover body is positioned between the first cavity and the second cavity.

19. A device according to claim 18, wherein the first cavity forces the movable element of the collector to be separated away from the through hole on the cover body.

20. A device according to claim 19, wherein when the cover body moves with respect to the collector, the sealed cavity on the collector, which is formed by the cover body and the collector, is compressed, the convex at the tip of the cover body touches and punctures the sealed film at the tip of the collecting rod, the sealed cavity is communicated with a hollow cavity of the collecting rod.

21. A device according to claim 20, wherein the cover body forces the first convex on the rib of the first cavity to be separated away from the through hole of the second cavity.

22. A device according to claim 21, wherein the rib of the first cavity comprises a second convex, the cover body forces the second convex to make the rib be retracted; after the rib is retracted, the first convex on the rib is separated away from the through hole in the second cavity.

23. A device according to claim 22, wherein when the sample collector together with the cover body is rotated in the first cavity and the second cavity, the suction element is compressed in the first cavity, the liquid flows into the first cavity and the second cavity; the first cavity together with the cover body and the collector is moved and reaches the first position of the second cavity, the first cavity seals the second cavity and forms the sealed cavity.

24. A device according to claim 23, wherein when the first cavity together with the cover body is rotated and reaches the second position of the second cavity, the sealed cavity is compressed, the liquid in the sealed cavity enters into the test cavity quantitatively.

25. A device according to claim 18, wherein the outer surface of the cover body and the inner surface of the second cavity have inter-occlusive threads.

26. A method for collecting a sample, wherein the method provides a device for collecting a sample, the device comprises a first cavity and a second cavity, the first cavity is positioned in the second cavity, both of the first cavity and the second cavity are separably connected and in liquid communication; the first cavity may seal and compress the second cavity so that the liquid in the second cavity may be discharged out quantitatively.

27. A method according to claim 26, wherein the first cavity and the second cavity may be rotated with each other, so, the first cavity is rotated and reaches a first position of the second cavity, the first cavity seals the second cavity.

28. A method according to claim 27, wherein the first cavity is rotated and reaches a second position of the second cavity, the sealed second cavity is compressed so that the liquid in the sealed second cavity is discharged out quantitatively.

29. A method according to claim 28, wherein the device still comprises a sample collector and a cover body which are movably connected; the sample collector comprises a hollow collecting rod, the tip of the collecting rod is sealed with a sealed film, and the end thereof is connected with and communicated with a suction element; the cover body and the tip of the collecting rod form a sealed cavity on the collector.

30. A method according to claim 29, wherein the sample collector and the cover body which are fixedly connected are inserted into the first cavity and the second cavity; the cover body is rotated so that the first cavity compresses a movable element on the sample collector to force the sample collector and the cover body to be separated away from fixed connection.

31. A method according to claim 30, wherein the cover body is rotated again so that the cover body and the sample collector are rotated with each other, the sealed cavity on the collector is compressed, the convex at the tip of the cover body touches and punctures the sealed film at the tip of the collecting rod, the sealed cavity is communicated with a hollow cavity of the collecting rod; a buffer solution in the sealed cavity flows onto the suction element through the communicated hollow cavity.

32. A method according to claim 31, wherein the cover body is rotated again so that the cover body compresses a rib on the first cavity to force the first cavity connected separably to be separated away from the second cavity.

33. A method according to claim 32, wherein the cover body is rotated again so that the cover body together with the sample collector is rotated in the first cavity and the second cavity, the suction element is compressed in the first cavity, the liquid flows into the first cavity and the second cavity; the first cavity together with the cover body and the collector is moved and reaches the first position of the second cavity, the first cavity seals the second cavity and forms the sealed cavity.

34. A method according to claim 33, wherein the cover body is rotated again so that the first cavity within the second cavity is moved and reaches the second position, the sealed cavity is compressed, the liquid in the sealed cavity quantitatively enters into a test cavity communicated with the sealed cavity.

35. A device for collecting a sample, wherein the device comprises a sample collector, a sealed cavity is provided on the collector.

36. A device according to claim 35, wherein the sealed cavity may be in liquid communication with a suction element on the collector through a passage.

37. A device according to claim 36, wherein the device still comprises a cover body, the cover body and the collector form the sealed cavity on the collector.

38. A device according to claim 37, wherein the cover body is separably connected with the collector.

39. A device according to claim 38, wherein the cover body and the collector, through a movable element on the collector, are matched with the through hole of the cover body, forming fixed connection and separation.

40. A device according to claim 39, wherein when the movable element is positioned in a hole on the cover body, the cover body is fixedly connected with the collector; when the movable element is separated away from the hole on the cover body, the cover body and the collector are movable between them.

41. A device according to claim 40, wherein the volume of the sealed cavity, through the movement between the cover body and the collector, is decreased or increased.

42. A device according to claim 40, wherein with the movement between the cover body and the collector, the sealed cavity and the suction element of the collector are communicated with each other through the passage.

43. A device according to claim 35, wherein the passage comprises a hollow collecting rod, one end of the collecting rod is connected with the suction element, and the other end of the collecting rod is sealed and connected with the sealed cavity.

44. A device according to claim 43, wherein a sealed film at the tip of the collecting rod is punctured with a convex in the sealed cavity, so that the sealed cavity and the hollow collecting rod are communicated with the suction element.

45. A device according to claim 35, wherein a buffer solution is provided in the sealed cavity.
